(19) United States
(12) Plant Patent Application Publication
(10) Pub. No.: US 2011/0197323 P1
(43) Pub. Date: Aug. 11, 2011

(54) Hybrid tea rose plant named 'Meitelov'

(50) ;

(75) Inventor: Alain A. Meilland, Antibes (FR)

(73) Assignee: CP DELAWARE, INC., Wilmington, DE (US)

(21) Appl. No.: 13/064,778

(22) Filed: Apr. 14, 2011

Related U.S. Application Data
(63) Continuation of application No. 12/805,046, filed on Jul. 8, 2010.
Application No. 12/805,046 is a continuation of application No. 12/385,490, filed on Apr. 9, 2009, now abandoned.
Application No. 12/385,490 is a continuation of application No. 11/505,878, filed on Aug. 18, 2006, now abandoned.
Application No. 11/505,878 is a continuation of application No. 11/062,548, filed on Feb. 23, 2005, now abandoned.
Application No. 11/062,548 is a continuation of application No. 10/777,101, filed on Feb. 13, 2004, now abandoned.
Application No. 10/777,101 is a continuation of application No. 10/445,017, filed on May 27, 2003, now abandoned.
Application No. 10/445,017 is a continuation of application No. 09/649,218, filed on Aug. 28, 2000, now abandoned.

(51) Int. Cl. A01H 5/00

(52) U.S. Cl. PLT/134

(57) ABSTRACT

A new and distinct variety of Hybrid Tea rose plant is provided which abundantly forms attractive large double yellow blossoms that are lightly perfumed. The plant exhibits a strong vigorous growth habit, semi-glossy decorative dark green foliage, and excellent disease resistance. The attractive dark green foliage contrasts nicely with the yellow blossoms. The new variety is particularly well suited for growing as attractive ornamentation in the landscape such as in parks and gardens.

This is a Continuation of U.S. Plant patent application Ser. No. 12/805,046, filed Jul. 8, 2010, which is a Continuation of application Ser. No. 12/385,490, filed Apr. 9, 2009, which is a Continuation of U.S. Plant patent application Ser. No. 11/505,878, filed Aug. 18, 2006 (now abandoned), which is a Continuation of U.S. Plant patent application Ser. No. 11/062,548, filed Feb. 23, 2005 (now abandoned), which is a Continuation of U.S. Plant patent application Ser. No. 10/777,101, filed Feb. 13, 2004 (now abandoned), which is a Continuation of U.S. Plant patent application Ser. No. 10/445,017, filed May 27, 2003 (now abandoned), which is a Continuation of U.S. Plant patent application Ser. No. 09/649,218, filed Aug. 28, 2000 (now abandoned).

### BOTANICAL/COMMERCIAL CLASSIFICATION

Rosa hybrida/Hybrid Tea Rose

### VARIETAL DENOMINATION

cv. Meitelov

### SUMMARY OF THE INVENTION

The new variety of Rosa hybrida Hybrid Tea rose plant was created by artificial pollination wherein two parents were crossed which previously had been studied in the hope that they would contribute the desired characteristics. The female parent (i.e., the seed parent) of the new variety was formed by crossing the ‘Meidragelac’ variety (non-patented in the United States) and the ‘Meikinosi’ variety (non-patented in the United States). The male parent (i.e., the pollen parent) was the ‘Korbelma’ variety (non-patented in the United States). The parentage of the new variety can be summarized as follows:
(‘Meidragelac’×‘Meikinosi’)×‘Korbelma’.

The seeds resulting from the above pollination were sown and small plants were obtained which were physically and biologically different from each other. Selective study resulted in the identification of a single plant of the new variety.

It was found that the new variety of Hybrid Tea rose plant of the present invention possesses the following combination of characteristics:
(a) exhibits an erect growth habit,
(b) exhibits a strong vigorous growth habit,
(c) abundantly forms over substantially the entire season attractive large double yellow blossoms that are lightly perfumed,
(d) forms decorative dark green semi-glossy foliage that contrasts well with the yellow blossoms, and
(e) is particularly well suited for growing as attractive ornamentation in the landscape.

The disease resistance of the new variety is excellent.

The new variety of the present invention can be readily distinguished from its ancestors. More specifically, the ‘Meidragelac’ variety displays a shorter growth habit and bears bicolored salmon flowers, the ‘Meikinosi’ variety displays a shorter growth habit and bears flowers having a lesser number of petals, and the ‘Korbelma’ variety displays dissimilar high-pointed buds and flowers having a lesser number of petals.

The new variety well meets the needs of the horticultural industry and can be grown to advantage in parks and gardens.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The initiation of rooting on cuttings takes place in an expeditious manner. For instance, when semi-hardwood cuttings are placed in the greenhouse during July, new plants commonly can be planted in the field during the second week of September.

The new variety has been named the ‘Meitelov’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were one year of age and were observed during September while budded on Rosa froebelli understock and growing in outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface; and

FIG. 16—illustrates a specimen of a leaf with seven leaflets—plan view—upper surface.

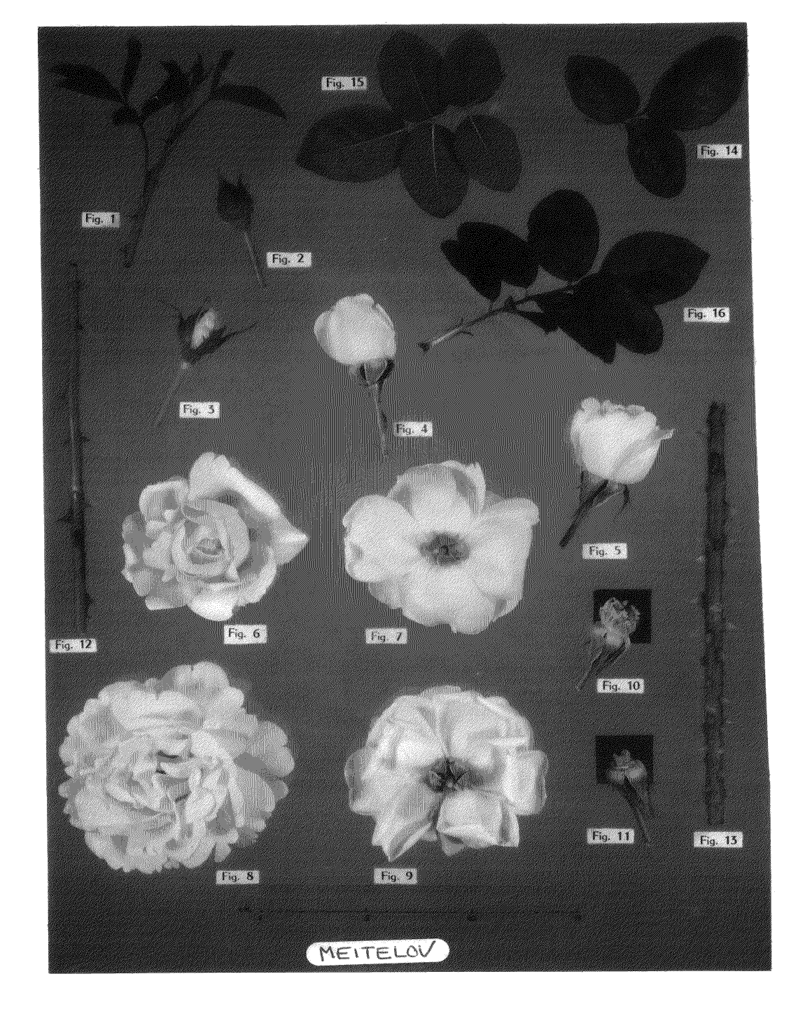

### DETAILED DESCRIPTION

The chart used in the identification of the colors is that of The Royal Horticultural Society (R.H.S. Colour Chart). The description is based on the observation of one year-old plants during October while budded on Rosa froebelli understock and growing outdoors at Le Cannet des Maures, Var, France. The coloration in more common terms precedes reference to the chart in some instances. Such terminology is to be accorded its ordinary dictionary significance.
Class: Hybrid Tea.
Plant:
   Height.—approximately 120 to 130 cm at the end of the growing season.
   Width.—approximately 90 to 100 cm at the end of the growing season.
   Habit.—erect.
Branches:
   Color.—young stems: near Green Group 143A. — adult wood: near Green Group 137A.
   Diameter.—commonly approximately 1.3 cm.
   Thorns.—size: large and strong (as illustrated), and commonly approximately 1 cm in length. — quantity: moderately numerous (as illustrated). On 10 cm of mature wood commonly approximately 15 thorns are present on average. — color: pale green (near Yellow-Green Group 144B) on young stems and tan (near Greyed-Orange Group 173B) on adult wood. — configuration: concave.
Leaves:
   Stipules.—adnate, pectinate, approximately 2.6 cm in length, and approximately 0.6 cm in width. The upper surface is near Green Group 136C and the under surface is near Green Group 137B.
   Size.—Approximately 6.8 cm in length on average, and approximately 3.8 cm in width on average.
   Petioles.—upper surface: near Green Group 131A. — under surface: near Green Group 138B. — texture: glandular on upper surface and bear a few prickles on under surface. — length: Approximately 1.2 cm on average on terminal leaflet. — diameter: commonly approximately 0.2 cm on average.
   Leaflets.—number: 3, 5 (most often), and 7. — shape: oval with a pointed apex and obtuse at the base. — size: commonly approximately 5 cm in length and approximately 4 cm in width on average. — serration: simple and regular (as illustrated). — texture: smooth and leathery. — general appearance: very dense, medium green, and semi-dull. — color (young foliage): upper surface: near Green Group 131A. under surface: lighter green, near Green Group 137A. — color (adult foliage): upper surface: near Green Group 131A. under surface: near Green Group 137A. — venation: typical for the leaves of the rose plant and near Green Group 138B in coloration.
Inflorescence:
   Number of flowers.—usually one to three flowers per stem.
   Peduncle.—rigid, medium in thickness, Yellow-Green Group 144A, the diameter commonly is approximately 0.4 cm on average, and the length is approximately 4 cm on average.
   Sepals.—upper surface: five in number, tomentose and near Green Group 138A and 138B. The sepals commonly are approximately 2 cm in length and approximately 0.8 cm in width. — under surface: near Green Group 143A with weak extensions (as illustrated).
   Buds.—shape: conical. — size: large. — length: approximately 3.5 cm on average. — diameter: approximately 2 cm on average. — color upon opening: upper surface: Lemon Yellow, near Yellow-Orange Group 14C, and widely suffused with Yellow-Orange Group 14B. under surface: Lemon Yellow, near Yellow-Orange Group 14C, and widely suffused with Yellow-Orange Group 14B.
   Flower.—shape: cup-shaped. — diameter: approximately 11 to 13 cm on average. — color (when opening begins): upper surface: Lemon Yellow, near Yellow-Orange Group 14C, and widely suffused with Yellow-Orange Group 14B. under surface: near Yellow-Orange Group 14C, and widely suffused with Yellow-Orange Group 14B. — color (when blooming): upper surface: Lemon Yellow, near Yellow-Orange Group 14C and widely suffused with Yellow-Orange Group 14B. under surface: Lemon Yellow, near Yellow-Orange Group 14C and widely suffused with Yellow-Orange Group 14B. — color (at end of opening): upper surface: near Yellow Group 8A and suffused with Yellow Group 8B. under surface: near Yellow Group 8A and suffused with Yellow Group 8B. — fragrance: light. — lasting quality: very long on the plant. The blossoms commonly last approximately 8 to 10 days on the plant depending upon environmental conditions. — petal shape: denticulate with a rounded apex and a wedge-shaped base. — petal size: on an open flower commonly approximately 4 cm in length and approximately 3.8 cm in width. — petal texture: soft. — petaloids: none observed during observations to date. — stamen number: approximately 62 on average. — anthers: approximately 0.3 cm in size and near Yellow-Orange Group 21C in coloration. — pollen: generally yellow in coloration. — filaments: approximately 1.1 cm in length on average and near Yellow-Orange Group 21A in coloration. — pistils: approximately 75 on average. — stigmas: near Orange Group 26A in coloration. — styles: approximately 0.6 cm in length on average and approximately 75 on average. — receptacle: medium green, near Green Group 143A in coloration, and pitcher-shaped in longitudinal section. — hips: pitcher-shaped in longitudinal section, approximately 1.2 cm in size, and near Greyed-Orange Group 168B in coloration. — seeds: generally round, approximately 0.2 cm in size on average, and commonly number approximately 18 on average per hip.
Development:
   Vegetation.—very vigorous and strong. When pruned to a height of 35 cm, a height of approximately 120 to 130 commonly is displayed at the end of the growing season.
   Blooming.—very abundant.
   Hardiness.—has withstood temperatures above −10° C.
   Resistance to diseases.—excellent with respect to Oidium and Black Spot.
   Aptitude to bear fruit.—good.

I claim:

1. A new and distinct variety of Hybrid Tea rose plant characterized by the following combination of characteristics:
(a) exhibits an erect growth habit,
(b) exhibits a strong vigorous growth habit,
(c) abundantly forms over substantially the entire season attractive large double yellow blossoms that are lightly perfumed,
(d) forms decorative dark green semi-glossy foliage that contrasts well with the yellow blossoms, and
(e) is particularly well suited for growing as attractive ornamentation in the landscape;
substantially as herein shown and described.
